(19) United States
(12) Patent Application Publication
(10) Pub. No.: US 2024/0192157 A1
(43) Pub. Date: Jun. 13, 2024

(54) METHODS AND SYSTEMS FOR DETERMINING COMBUSTION CHARACTERISTICS OF MIXTURES OF GASIFICATION SLAG AND COAL

(71) Applicant: CHINA UNIVERSITY OF MINING AND TECHNOLOGY, Xuzhou (CN)

(72) Inventors: Yixin ZHANG, Xuzhou (CN); Yan LI, Xuzhou (CN); Jianjun WU, Xuzhou (CN); Fanhui GUO, Xuzhou (CN); Wenke JIA, Xuzhou (CN); Yang GUO, Xuzhou (CN); Hongguan WANG, Xuzhou (CN); Sixi GUO, Xuzhou (CN)

(73) Assignee: CHINA UNIVERSITY OF MINING AND TECHNOLOGY, Xuzhou (CN)

(21) Appl. No.: 18/467,717

(22) Filed: Sep. 14, 2023

(30) Foreign Application Priority Data
Dec. 7, 2022 (CN) 202211561589.3

(51) Int. Cl. G01N 25/22; G01N 33/24

(52) U.S. Cl. CPC G01N 25/22; G01N 33/24

(57) ABSTRACT

The present disclosure provides methods for determining a combustion characteristic of a mixture of gasification slag and coal. The method may be implemented by a processor. The combustion characteristic includes at least one of an ignition temperature, a burnout temperature, or a composite combustion characteristic index. The method comprises: obtaining first basic data of sample coal and sample gasification slag, the first basic data including at least one of a moisture content, a volatile matter, or a fixed carbon; obtaining second basic data of coal to be tested and gasification slag to be tested in a mixture to be tested and a mixing ratio of the mixture to be tested; and determining a combustion characteristic of the mixture to be tested based on the first basic data, the second basic data, and the mixing ratio.

### CROSS-REFERENCE TO RELATED APPLICATIONS

This application claims the priority of the Chinese Patent Application No. 202211561589.3, filed on Dec. 7, 2022, entitled “METHODS FOR CALCULATING COMBUSTION CHARACTERISTICS OF MIXTURE OF GASIFICATION SLAG AND COAL IN DIFFERENT RATIOS,” the entire contents of which are incorporated herein by reference.

### TECHNICAL FIELD

The present disclosure relates to the technical field of coal combustion, and in particular, to methods and systems for determining a combustion characteristic of a mixture of gasification slag and coal.

### BACKGROUND

Gasification, as one of the significant means for clean and efficient utilization of coal, has become a key focus of the field of coal chemical industry. However, a large amount of gasification slag is generated during the gasification, which has a high ignition loss and carbon content. Currently, the most common processing approach is landfill or stacking. However, this approach not only occupies land resources but also causes pollution to the soil, surface water, and even groundwater

A considerable number of experiments have shown that co-combusting gasification slag with coal in a certain proportion may achieve a certain degree of synergy. Therefore, blending combustion of gasification slag and coal is considered a feasible technical solution for resource utilization. A combustion characteristic plays an important reference value for characterizing an actual combustion situation of fuel in a boiler, which is a significant part of fuel combustion theory. The combustion characteristic of a mixture provides guiding significance for the application of different mixed raw materials and mixed fuels with different ratios in boilers.

However, it is not practical for conducting a plurality of tests on combustion effects of different mixed fuels in industrial boilers. In current research, the effect of blending combustion of gasification slag and coal is mainly analyzed through thermogravimetric analysis, which has a high requirement for an experimenter and a testing instrument. Moreover, a plurality sets of tests force experimental personnel into repetitive work, which wastes time and manpower.

Therefore, it is desirable to provide methods and systems for determining a combustion characteristic of a mixture of gasification slag and coal to achieve a rapid, efficient, and accurate determination of the combustion characteristic of the mixture.

### SUMMARY

One or more embodiments of the present disclosure provide a method for determining a combustion characteristic of a mixture of gasification slag and coal, implemented by a processor. The combustion characteristic includes at least one of an ignition temperature, a burnout temperature, or a composite combustion characteristic index. The method includes: obtaining first basic data of sample coal and sample gasification slag, the first basic data including at least one of a moisture content, a volatile matter, or a fixed carbon; obtaining second basic data of coal to be tested and gasification slag to be tested in a mixture to be tested and a mixing ratio of the mixture to be tested; and determining a combustion characteristic of the mixture to be tested based on the first basic data, the second basic data, and the mixing ratio.

One or more embodiments of the present disclosure provide a system for determining a combustion characteristic of a mixture of gasification slag and coal. The combustion characteristic includes at least one of an ignition temperature, a burnout temperature, or a composite combustion characteristic index. The system includes: a first obtaining module configured to obtain first basic data of sample coal and sample gasification slag, the first basic data including at least one of a moisture content, a volatile matter, or a fixed carbon; a second obtaining module configured to obtain second basic data of coal to be tested and gasification slag to be tested in a mixture to be tested and a mixing ratio of the mixture to be tested; and a determination module configured to determine a combustion characteristic of the mixture to be tested based on the first basic data, the second basic data, and the mixing ratio.

One or more embodiments of the present disclosure provide a non-transitory computer-readable storage medium storing computer instructions. When reading the computer instructions in the storage medium, a computer executes the method for determining a combustion characteristic of a mixture of gasification slag and coal according to any of the embodiments.

### BRIEF DESCRIPTION OF THE DRAWINGS

The present disclosure will be further illustrated by way of exemplary embodiments, which will be described in detail by way of the accompanying drawings. These embodiments are not limiting, and in these embodiments the same numbering indicates the same structure, wherein:

FIG. 1 is a flowchart illustrating an exemplary a process of a method for determining a combustion characteristic of a mixture of gasification slag and coal according to some embodiments of the present disclosure;

FIG. 2 is an exemplary schematic diagram illustrating determining a combustion characteristic of a mixture to be tested according to some embodiments of the present disclosure;

FIG. 3 is a flowchart illustrating an exemplary process for determining a correction coefficient according to some embodiments of the present disclosure;

FIG. 4 is a schematic diagram illustrating an exemplary first coefficient determination model according to some embodiments of the present disclosure;

FIG. 5 is a schematic diagram illustrating an exemplary second coefficient determination model according to some embodiments of the present disclosure; and

FIG. 6 is a schematic diagram illustrating an exemplary combustion characteristic prediction model according to some embodiments of the present disclosure.

### DETAILED DESCRIPTION

In order to more clearly illustrate the technical solutions of the embodiments of the present disclosure, the following will be a brief description of the accompanying drawings that need to be used in the description of the embodiments. It will be apparent that the accompanying drawings in the following description are only examples or embodiments of the present disclosure, and that other similar scenarios may be applied to the present disclosure by those of ordinary skill in the art, without creative effort. Unless obviously obtained from the context or the context illustrates otherwise, the same numeral in the drawings refers to the same structure or operation.

It should be understood that the terms “system,” “device,” “unit,” and/or “module” used in the present disclosure are used to distinguish different components, elements, parts, or assemblies at different levels. However, if other terms may achieve the same purpose, they may be replaced with other expressions.

As shown in the present disclosure and the claims, unless the context clearly indicates otherwise, the terms “one,” “a,” “an,” “one kind,” and/or “the” are not limited to singular and may also include plural. Generally, the terms “including” and “comprising” merely indicate the inclusion of specifically identified steps and elements, and these steps and elements do not constitute an exclusive listing. The methods or devices may also include other steps or elements.

Flowcharts are used throughout the present disclosure to illustrate the operations performed by the system according to embodiments of the present disclosure. It should be understood that the preceding or following operations are not necessarily performed in exact order. Instead, the individual steps may be processed in reverse order or simultaneously. It is also possible to add other operations to these procedures or remove a step or steps from them.

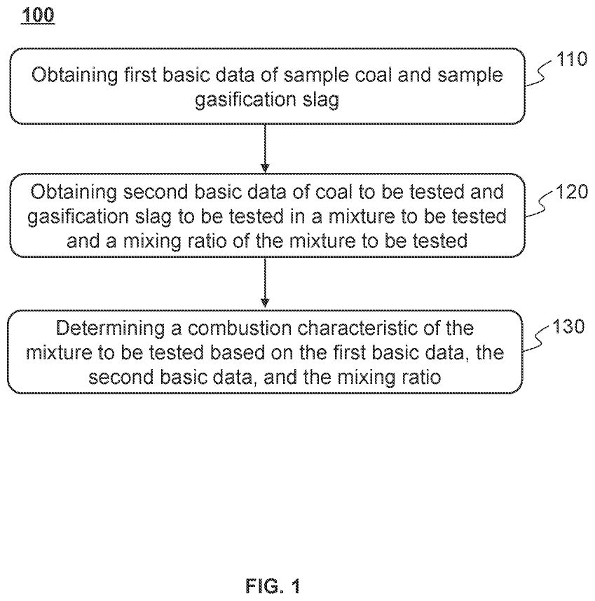

FIG. 1 is a flowchart illustrating an exemplary a process of a method for determining a combustion characteristic of a mixture of gasification slag and coal according to some embodiments of the present disclosure. In some embodiments, the process 100 may be executed by a processor. As shown in FIG. 1, the process 100 may include the operations 110, 120, and 130.

In 110, obtaining first basic data of sample coal and sample gasification slag.

The sample coal refers to coal used as an experimental sample.

The sample gasification slag refers to gasification slag used as an experimental sample.

The first basic data refers to data related to a composition and a property of the sample coal or the sample gasification slag. In some embodiments, the first basic data may include at least one of a moisture content M, a volatile matter V, or a fixed carbon FC. In some embodiments, the first basic data may also include other basic data, such as an ash content A, a calorific value Q, a combustion characteristic, etc. More descriptions about the combustion characteristic may be found in the operation 130 and the related description thereof.

In some embodiments, a storage unit may pre-store corresponding relationships between different varieties of coal or gasification slag and the first basic data. The processor may obtain the first basic data of the sample coal or the sample gasification slag through the corresponding relationship by accessing the storage unit based on a determined variety of the sample coal or the sample gasification slag.

In some embodiments, a first quantity and variety of coal may be selected as the sample coal and a second quantity and variety of gasification slag may be selected as the sample gasification slag. The processor may determine the first basic data of the sample coal and the sample gasification slag by controlling one or more testing units to perform at least one of an industrial analysis, an elemental analysis, a calorific value, and combustion characteristic test on the sample coal and the sample gasification slag. Further, the processor may establish a basic database of the sample coal and the sample gasification slag based on the first basic data and store the basic database in one or more storage units.

In some embodiments, the first quantity and variety and the second quantity and variety may be default values or preset values.

In some embodiments, the industrial analysis may include determining the moisture content M, the ash content A, the volatile matter V, and the fixed carbon FC. For example, the industrial analysis may be performed according to the GB212-91 standard.

In some embodiments, the elemental analysis may include determining carbon C, hydrogen H, oxygen O, nitrogen N, and sulfur S. For example, the elemental analysis may be performed according to the GB/T476 standard.

In some embodiments, the calorific value is tested following the GB213-87 standard, and the combustion characteristic test is performed using a thermogravimetric analyzer.

In some embodiments, the processor may establish the basic database of the sample coal or the sample gasification slag based on each variety of sample coal or each variety of sample gasification slag and the first basic data corresponding to the sample coal or the sample gasification slag, and store the basic database in one or more storage units.

Merely by way of example, in some embodiments, the sample coal and/or sample gasification slag may be selected based on different gasifiers that produce gasification slag and coal with different coalification levels. The sample gasification slag may be produced from a Texaco gasifier, a Siemens gasifier (GSP furnace), a Shell gasifier, a water gasifier (UGI furnace), and a Shenning gasifier, respectively. Each gasifier is sampled twice in a continuous working process, and a result of each sampling is taken as a variety of sample gasification slag, and a second sampling is performed at an interval of 30 days since a first sampling for each gasifier. The sample coal may be anthracite, coking coal, bituminous coal, gas coal, and lignite, respectively. The processor may number 10 varieties of selected sample gasification slag as C₁, C₂, . . . , C₁₀; and number 5 varieties of selected sample coal as G₁, G₂, . . . , G₅.

In some embodiments of the present disclosure, the first basic data may be determined by performing tests on the sample coal and the sample gasification slag using the testing units, which can improve the accuracy of determining the first basic data. The basic database of the sample coal and the sample gasification slag may be established and the basic database may be stored in the storage unit, which make it convenient to obtain the first basic data of the sample coal and the sample gasification slag subsequently by directly accessing the storage unit.

In 120, obtaining second basic data of coal to be tested and gasification slag to be tested in a mixture to be tested and a mixing ratio of the mixture to be tested.

The mixture to be tested refers to a mixture that requires the combustion characteristic test. The mixture to be tested may be obtained by mixing an unknown variety of coal to be tested (referred to as coal to be tested m hereafter) and an unknown variety of gasification slag to be tested (referred to as gasification slag to be tested n hereafter).

The second basic data refers to data related to a composition and a property of the coal to be tested or the gasification slag to be tested.

In some embodiments, the processor may determine the second basic data of the coal to be tested and the gasification slag to be tested by controlling one or more testing units to perform at least one of the industrial analysis, the elemental analysis, a calorific value, and combustion characteristic test on the coal to be tested and the gasification slag to be tested. The processor determines the second basic data in a similar manner to the manner that the processor determines the first basic data. For information on how the processor determines the second basic data, please refer to the manner that the processor determines the first basic data.

In some embodiments of the present disclosure, the second basic data may be determined by performing tests on the coal to be tested and the gasification slag to be tested using the testing units, which can improve the accuracy of determining the second basic data.

The mixing ratio refers to a mass ratio of the coal to be tested to the gasification slag to be tested in the mixture to be tested.

In some embodiments, the processor may separate the coal to be tested and the gasification slag to be tested in the mixture to be tested, measure a mass of the separated coal to be tested and a mass of the gasification slag to be tested using a weighing device, calculate and determine the mixing ratio of the coal to be tested and the gasification slag to be tested. The coal to be tested and the gasification slag to be tested in the mixture to be tested may be separated in various ways, such as a flotation manner, an extraction manner, etc.

In 130, determining a combustion characteristic of the mixture to be tested based on the first basic data, the second basic data, and the mixing ratio.

The combustion characteristic refers to a characteristic related to the combustion of fuel. For example, the combustion characteristic may include at least one of an ignition temperature T, a burnout temperature t, and a composite combustion characteristic index B. The ignition temperature refers to a minimum temperature at which the fuel ignites and continues to burn in an air or oxygen atmosphere. The burnout temperature refers to a temperature at which 98% of combustible components of the fuel are burned. The composite combustion characteristic index refers to a parameter that reflects the ignition and burnout performance of the fuel.

In some embodiments, the processor may calculate a similarity between the second basic data and each piece of first basic data, determine sample coal and sample gasification slag corresponding to first basic data with a highest similarity, calculate differences between each basic data of the sample coal and each basic data of the sample gasification fine slag and each basic data in the second basic data, determine a combustion coefficient by performing a weighted summation on the differences of each basic data, and determine the combustion characteristic of the mixture to be tested based on the combustion coefficient and the mixing ratio.

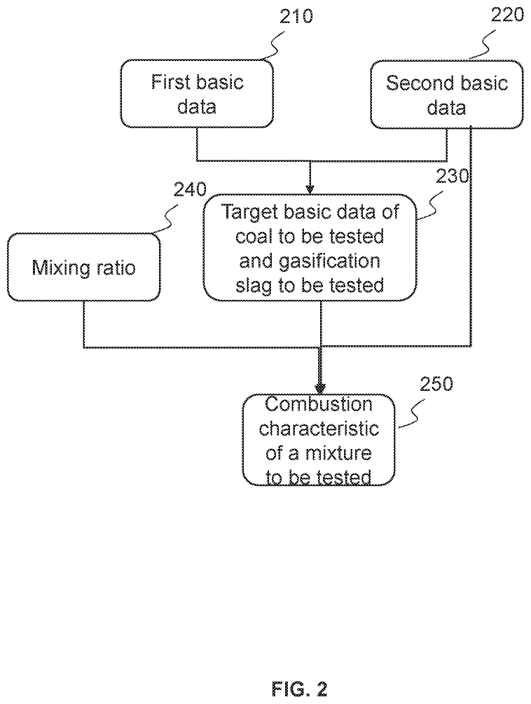

In some embodiments, the processor may determine the target basic data of the coal to be tested and the gasification slag to be tested based on the first basic data and the second basic data, and determine the combustion characteristic of the mixture to be tested based on the target basic data, the second basic data, and the mixing ratio of the coal to be tested and the gasification slag to be tested in the mixture to be tested. More descriptions about the embodiment may be found in FIG. 2 and the relevant descriptions thereof.

In some embodiments of the present disclosure, the combustion characteristic of the mixture to be tested may be determined based on the first basic data of the sample coal and the sample gasification slag, the second basic data of the coal to be tested and the gasification slag to be tested, and the mixing ratio of the coal to be tested and the gasification slag to be tested in the mixture to be tested, which is conducive to determining the combustion characteristic of the mixture to be tested quickly, efficiently, and accurately.

FIG. 2 is an exemplary schematic diagram illustrating determining a combustion characteristic of a mixture to be tested according to some embodiments of the present disclosure.

In some embodiments, the processor may determine target basic data 230 of coal to be tested and gasification slag to be tested based on first basic data 210 and second basic data 220, and determine the combustion characteristic 250 of a mixture to be tested based on the target basic data, the second basic data 220, and a mixing ratio 240.

More descriptions about the first basic data, the second basic data, the mixing ratio, and the combustion characteristic may be found in FIG. 1 and the relevant descriptions.

The target basic data refers to a set of basic data of the first basic data 210 that is most similar to the second basic data 220.

In some embodiments, the processor may determine the target basic data of the coal to be tested and the gasification slag to be tested in various ways based on the first basic data 210 and the second basic data 220. For example, the processor may calculate a sum of squares f of differences between the second basic data corresponding to the coal to be tested and the first basic data corresponding to each variety of sample coal in the basic database. The processor may determine first basic data of sample coal with a smallest sum of squares f as the target basic data of the coal to be tested.

As another example, the processor may calculate a sum of squares h of differences between the second basic data corresponding to the gasification slag to be tested and the first basic data corresponding to each variety of sample gasification slag in the basic database. The processor may determine first basic data of sample gasification slag with a smallest sum of squares h as the target basic data of the gasification slag to be tested.

In some embodiments, experimental samples providing the two sets of target basic data are also referred to as target basic samples. The target basic sample corresponding to the coal to be tested m is denoted as target coal α, and the target basic sample corresponding to the gasification slag to be tested n is denoted as target gasification slag β.

Merely by way of example, various basic data of the first basic data and the second basic data may be represented as follows: a moisture content Mm, an ash content Aₘ, a volatile matter Vₘ, carbon Cₘ, hydrogen Hₘ, oxygen Oₘ, nitrogen Nₘ, sulfur Sₘ, and a calorific value Qₘ for coal; a moisture content Mₙ, an ash content Aₙ, a volatile matter Vₙ, carbon Cₙ, hydrogen Hₙ, oxygen Oₙ, nitrogen Nₙ, sulfur Sₙ, and a calorific value Qₙ for gasification slag.

Taking the moisture content M as an example, the moisture content corresponding to each variety of sample gasification slag in the basic database may be represented as M_{C}_{₁}, M_{C}_{₂}, . . . , M_{C}_{₁₀}, and the moisture content corresponding to each variety of sample coal may be represented as M_{G}_{₁}, M_{G}_{₂}, . . . , M_{G}_{₅}. Other data is represented in a similar way, which is not repeated herein.

In some embodiments, a process in which the processor determines the target coal α and the target gasification slag β may be expressed as follows.

For each variety of sample gasification slag, the processor may perform the following calculations:

f₁=(Mₙ−M_{C}_{₁})²+(Aₙ−A_{C}_{₁})²+ . . . +(Nₙ−N_{C}_{₁})²+(Sₙ−S_{C}_{₁})²,

f₂=(Mₙ−M_{C}_{₂})²+(Aₙ−A_{C}_{₂})²+ . . . +(Nₙ−N_{C}_{₂})²+(Sₙ−S_{C}_{₂})².

. . . , and

f₁₀=(Mₙ−M_{C}_{₁₀})²+(Aₙ−A_{C}_{₁₀})²+ . . . +(Nₙ−N_{C}_{₁₀})²+(Sₙ−S_{C}_{₁₀})².

The processor takes sample gasification slag corresponding to fₘᵢₙ=min{f₁, f₂, f₃, . . . , f₁₀} as the target basic sample. That is, the sample gasification slag is denoted as the target gasification slag β, and first basic data corresponding to the sample gasification is determined as the target basic data.

For each variety of sample coal, the processor may perform the following calculations:

h₁=(Mₘ−M_{G}_{₃})²+(Aₘ−A_{G}_{₁})²+ . . . +(Nₘ−N_{G}_{₁})²+(Sₘ−S_{G}_{₁})²

h₂=(Mₘ−M_{G}_{₂})²+(Aₘ−A_{G}_{₂})²+ . . . +(Nₘ−N_{G}_{₂})²+(Sₘ−S_{G}_{₂})²

. . . , and

h₅=(Mₘ−M_{G}_{₅})²+(Aₘ−A_{G}_{₅})²+ . . . +(Nₘ−N_{G}_{₅})²+(Sₘ−S_{G}_{₅})².

The processor takes sample coal corresponding to hₘᵢₙ=min {h₁, h₂, h₃, h₄, h₅} as the target basic sample. That is, the sample coal is denoted as the target coal a, and first basic data corresponding to the sample coal is determined as the target basic data.

In some embodiments, the processor may determine the combustion characteristic of the mixture to be tested based on the target basic data, the second basic data, and the mixing ratio, through a preset data comparison table. The preset data comparison table records combustion characteristics of different mixtures to be tested corresponding to different target basic data, second basic data, and mixing ratios. The comparison table may be preset based on prior knowledge or historical data.

In some embodiments, the processor may determine a first combustion coefficient Kₘ of the coal to be tested and a second combustion coefficient Kₙ of the gasification slag to be tested based on the target basic data and the second basic data, determine a combustion characteristic of the coal to be tested and a combustion characteristic of the gasification slag to be tested based on the first combustion coefficient, the second combustion coefficient, and the target basic data, and determine the combustion characteristic of the mixture to be tested based on the combustion characteristic of the coal to be tested, the combustion characteristic of the gasification slag to be tested, and the mixing ratio.

The first combustion coefficient Kₘ refers to a coefficient that reflects a relationship between the combustion characteristic of the coal to be tested m and the combustion characteristic of the target coal α.

The second combustion coefficient Kₙ refers to a coefficient that reflects a relationship between the combustion characteristic of the gasification slag to be tested n and the combustion characteristic of the target gasification slag β.

In some embodiments, the processor may determine the first combustion coefficient and the second combustion coefficient through a preset data comparison table based on the target basic data and the second basic data. The preset data comparison table records the first combustion coefficients and the second combustion coefficients corresponding to different target basic data and second basic data.

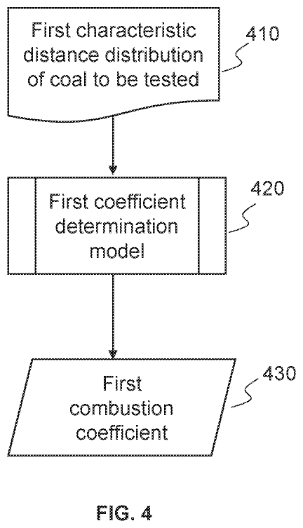

In some embodiments, the processor may determine the first combustion coefficient by processing a first characteristic distance distribution of the coal to be tested through a first coefficient determination model. More descriptions about the embodiment may be found in FIG. 4 and the relevant descriptions thereof.

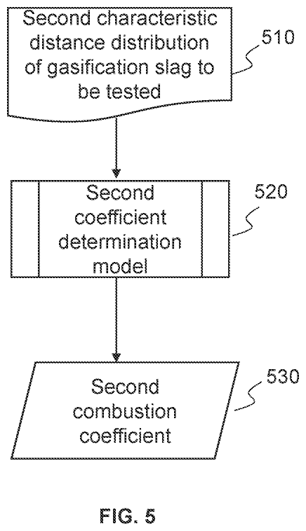

In some embodiments, the processor may determine the second combustion coefficient by processing a second characteristic distance distribution of the gasification slag to be tested through a second coefficient determination model. More descriptions about the embodiment may be found in FIG. 5 and the relevant descriptions thereof.

In some embodiments, the first combustion coefficient and the second combustion coefficient are related to a correction coefficient.

The correction coefficient refers to a coefficient that reflects a degree of impact of each piece of basic data on the combustion coefficient.

In some embodiments, the correction coefficients corresponding to a same type of basic data of the coal to be tested and the gasification slag to be tested are the same. For example, the correction coefficient of the moisture content of the second basic data of the coal to be tested is the same as the correction coefficient of moisture content of the second basic data of the gasification slag to be tested.

In some embodiments of the present disclosure, the correction coefficients corresponding to the same type of basic data of the coal to be tested and the gasification slag to be tested are set to be a same value, which not only makes the setting of the correction coefficient more reasonable but also helps to reduce computation complexity.

In some embodiments, the processor may assign a value to the correction coefficient of each piece of basic data based on an importance of the each piece of basic data and confirm a validation range. For example, the processor may assign incremental values at an interval of 0.1 in a range of 0-5 to the correction coefficients of the moisture content M, the ash content A, the volatile matter V, and the fixed carbon FC; assign incremental values at an interval of 0.1 in a range of 0-2 to the correction coefficients of the carbon C and the hydrogen H; and assign incremental values at an interval of 0.01 in a range of 0-1 to the correction coefficients of the oxygen O, the nitrogen N, the sulfur S, and the calorific value Q.

In some embodiments, the processor may randomly select two varieties of sample coal among the sample coal based on the basic database to conduct an orthogonal experiment according to respective values of the basic data, respectively, and determine a data set of the first combustion coefficients by calculation. The processor calculates an average of the data set of the first combustion coefficients, determines a first combustion coefficient Kₘ that is closest to the average of the data set of the first combustion coefficients, and determines a coefficient in an equation for calculating the first combustion coefficient Kₘ as the correction coefficient.

In some embodiments, the correction coefficient of the gasification slag to be tested may be the correction coefficient obtained by calculating based on the coal to be tested.

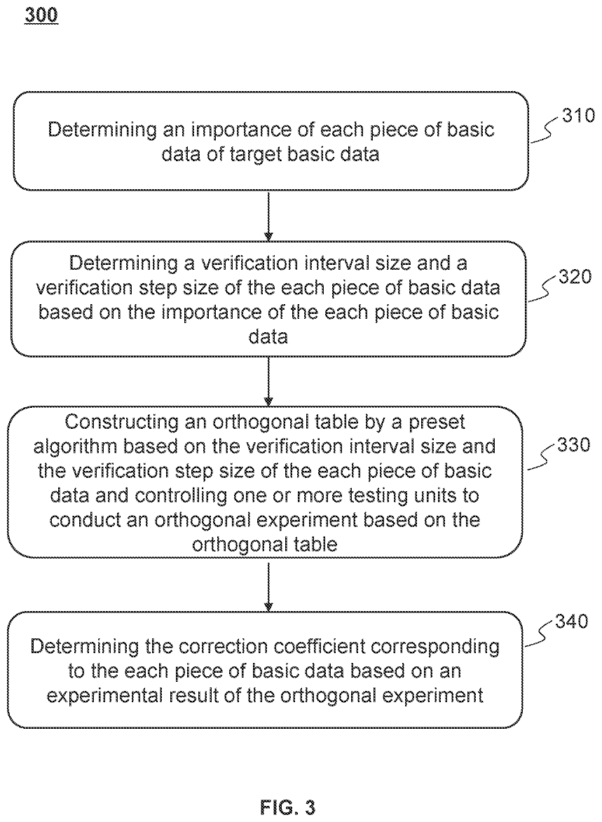

In some embodiments, the processor may determine an importance of each piece of basic data of the target basic data, determine a verification interval size and a verification step size of the each piece of basic data based on the importance of the each piece of basic data, construct an orthogonal table by a preset algorithm, and determine the correction coefficient corresponding to the each piece of basic data. More descriptions about the embodiment may be found in FIG. 3 and the relevant descriptions thereof.

In some embodiments, the first combustion coefficient of the coal to be tested and the second combustion coefficient of the gasification slag to be tested are positively correlated with the correction coefficient.

In some embodiments, the processor may calculate the first combustion coefficient Kₘ of the coal to be tested m based on the target basic data of the target coal α and the second basic data of the coal to be tested m. For example, the processor may determine the first combustion coefficient Kₘ by calculating a sum of squares of differences between basic data values of the target coal a and basic data values of the coal to be tested m and multiplying the squares by correction coefficients corresponding to various pieces of basic data, respectively.

In some embodiments, the processor may calculate the second combustion coefficient Kₙ of the gasification slag to be tested n based on the target basic data of the target gasification slag β and the second basic data of the gasification slag to be tested n. For example, the processor may determine the second combustion coefficient Kₙ by calculating a sum of squares of differences between basic data values of the target gasification slag β and basic data values of the gasification slag to be tested n and multiplying the squares by correction coefficients corresponding to various pieces of basic data, respectively.

Merely by way of example, in some embodiments, the correction coefficient of the moisture content M is 1, the correction coefficient of the ash content A is 1, the correction coefficient of the volatile matter V is 2, the correction coefficient of the fixed carbon FC is 3, the correction coefficient of the carbon C is 1.5, the correction coefficient of the hydrogen H is 1.5, the correction coefficient of the oxygen O is 0.05, the correction coefficient of the nitrogen N is 0.02, the correction coefficient of the sulfur S is 0.02, and the correction coefficient of the calorific value Q is 0.17.

In some embodiments, the equations for calculating the first combustion coefficient Kₘ and the second combustion coefficient Kₙ are as follows:

Kₘ=(M_{α}−Mₘ)²+2×(V_{α}−Vₘ)²+(A_{α}−Aₘ)²+3×(FC_{α}−FCₘ)²+1.5×(C_{α}−Cₘ)²+1.5×(H_{α}−Hₘ)²+0.05×(O_{α}−Oₘ)²+0.02×(N_{α}−Nₘ)²+0.02×(S_{α}−Sₘ)²+0.17×(Q_{α}−Qₘ)², and

Kₙ=(M_{β}−Mₙ)²+2×(V_{β}−Vₙ)²+(A_{β}−Aₙ)²+3×(FC_{β}−FCₙ)²+1.5×(C_{β}−Cₙ)²+1.5×(H_{β}−Hₙ)²+0.05×(O_{β}−Oₙ)²+0.02×(N_{β}−Nₙ)²+0.02×(S_{β}−Sₙ)²+0.17×(Q_{β}−Qₙ)².

In some embodiments of the present disclosure, the first combustion coefficient Kₘ and the second combustion coefficient Kₙ are related to the correction coefficient, and the first combustion coefficient and the second combustion coefficient are determined through the correction coefficient, which improves the accuracy of a determined first combustion coefficient and a determined second combustion coefficient. Moreover, the first combustion coefficient and the second combustion coefficient may be calculated using the same correction coefficient, which reduces the computational complexity.

In some embodiments, the processor may determine the combustion characteristics of the sample coal and the sample gasification slag through equations based on the first combustion coefficient, the second combustion coefficient, and the target basic data.

For example, an ignition temperature Tₘ of the coal to be tested m may be calculated as

$${T}_{m} = {T}_{α} \times \frac{{K}_{m}}{{L}_{m}} ,$$

where T_{α} denotes an ignition temperature of the target coal α, and Lₘ denotes a first ignition temperature coefficient. A burnout temperature tₘ of the coal to be tested m may be calculated as

$${t}_{m} = {t}_{α} \times \frac{{K}_{m}}{{R}_{m}} ,$$

where t_{α} denotes a burnout temperature of the target coal α, and Rₘ denotes a first burnout temperature coefficient. A composite combustion characteristic index Bₘ of the coal to be tested m may be calculated as

$${B}_{m} = {B}_{α} \times \frac{{K}_{m}}{{U}_{m}} ,$$

where B_{α} denotes a composite combustion characteristic index of the target coal α, and Uₘ denotes a first composite combustion coefficient.

An ignition temperature Tₙ of the gasification slag to be tested n may be calculated as

$${T}_{n} = {T}_{β} \times \frac{{K}_{n}}{{L}_{n}} ,$$

where T_{β} denotes an ignition temperature of the target gasification slag β, and Lₙ denotes a second ignition temperature coefficient. A burnout temperature tₙ of the gasification slag to be tested n may be calculated as

$${t}_{n} = {t}_{β} \times \frac{{K}_{n}}{{R}_{n}} ,$$

and t_{β} denotes a second burnout temperature coefficient. A composite combustion characteristic index Bₙ of the gasification slag to be tested n may be calculated as

$${B}_{n} = {B}_{β} \times \frac{{K}_{n}}{{U}_{n}} ,$$

where B_{β} denotes a composite combustion characteristic index of the target gasification slag β, and Uₙ denotes a second composite combustion coefficient.

In some embodiments, a manner for determining the first ignition temperature coefficient, the first burnout temperature coefficient, and the first composite combustion coefficient in the above equations is as follows: assigning values to the first ignition temperature coefficient, the first burnout temperature coefficient, and the first composite combustion coefficient based on linear experience. The first ignition temperature coefficient and the first burnout temperature coefficient are assigned with incremental values at an interval of 1 in a range of 1-100. The first composite combustion coefficient is assigned with incremental values at an interval of 10 in a range of 0-200.

In some embodiments, the processor performs a simulation prediction between any two varieties of sample coal, sums up deviations between predicted values and actual values, and determines a group of values with a smallest deviation in all simulation predictions as the first ignition temperature coefficient, the first burnout temperature coefficient, and the first composite combustion coefficient. A manner for determining the second ignition temperature coefficient, the second burnout temperature coefficient, and the second composite combustion coefficient is similar to the manner in the previous embodiment, which is not repeated herein.

In some embodiments, if the processor calculates the first ignition temperature coefficient to be 10, the first burnout temperature coefficient to be 15, the first combined combustion coefficient to be 110, the second ignition temperature coefficient to be 22, the second burnout temperature coefficient to be 24, and the second combined combustion coefficient to be 130 through the previously described manner, the equations for calculating the combustion characteristics of the sample coal and the sample gasification slag may be expressed as follows: for the coal to be tested m, the ignition temperature

$${T}_{m} = {T}_{α} \times \frac{{K}_{m}}{10} ,$$

the burnout temperature

$${t}_{m} = {t}_{α} \times \frac{{K}_{m}}{15} ,$$

and the composite combustion characteristic index

$${B}_{m} = {B}_{α} \times \frac{{K}_{m}}{110} ;$$

and for the gasification slag to be tested n, the ignition temperature

$${T}_{n} = {T}_{β} \times \frac{{K}_{n}}{22} ,$$

the burnout temperature

$${t}_{n} = {t}_{β} \times \frac{{K}_{n}}{24} ,$$

and the composite combustion characteristic index

$${B}_{n} = {B}_{β} \times \frac{{K}_{n}}{130} .$$

In some embodiments, the processor may determine the combustion characteristic of the mixture to be tested through equations based on the combustion characteristics of the sample coal and the sample gasification slag, and the mixing ratio.

For example, the processor may first calculate a mass fraction X of the coal to be tested m in the mixture to be tested based on the mixing ratio and calculate the combustion characteristic of the mixture to be tested through the equations.

For the mixture to be tested, the ignition temperature T=Dₘ×Tₘ×X+Dₙ×Tₙ×(1−X), the burnout temperature t=Eₘ×tₘ×X+Eₙ×tₙ×(1−X), and the composite combustion characteristic index B=Pₘ×Bₘ×X+Pₙ×Bₙ×(1−X), where Dₘ denotes a first mixed ignition temperature coefficient, Dₙ denotes a second mixed ignition temperature coefficient, Eₘ denotes a first mixed burnout temperature coefficient, Eₙ denotes a second mixed burnout temperature coefficient, Pₘ denotes a first mixed combustion characteristic index coefficient, and Pₙ denotes a second mixed combustion characteristic index coefficient.

In some embodiments, the first mixed ignition temperature coefficient, the second mixed ignition temperature coefficient, the first mixed burnout temperature coefficient, the second mixed burnout temperature coefficient, the first mixed combustion characteristic coefficient, and the second mixed combustion characteristic coefficient may be determined by the processor through calculation based on data of a large number of mixed fuels with different mixing ratios.

For example, the processor may utilize 10 varieties of sample gasification slag and 5 varieties of sample coal of the basic database to form 50 combinations of mixed fuels. These combinations of mixed fuels may be formed with five mixing ratios of 1/9, 3/7, 5/5, 7/3, and 9/1, resulting in a total of 250 mixed fuels. The processor randomly numbers the 250 mixed fuels and selects 10 mixed fuels for experimental testing, calculates the ignition temperature, the burnout temperature, and the composite combustion characteristic index for each of the 10 selected mixed fuels, which is used as standard data for calculating coefficients corresponding to the ignition temperature, the burnout temperature, and the composite combustion characteristic index.

Merely by way of example, taking the ignition temperature as an example, the processor sets coefficients of coal in a mixed sample to be incremental values at an interval of 0.02 in a range of 0.5-1.5 and sets coefficients of gasification slag to be incremental values at an interval of 0.02 in a range of 0.5-1.5. The processor performs a simulation prediction on the selected combinations sequentially and for each selected combination, calculates deviations between predicted values and experimental values, and finally determines a group of coefficients with a smallest summed deviation as the first mixed ignition temperature coefficient and the second mixed ignition temperature coefficient. A manner for determining the first mixed burnout temperature coefficient, the second mixed burnout temperature coefficient, the first mixed combustion characteristic coefficient, and the second mixed combustion characteristic coefficient is similar to the manner in the previous embodiment, which is not repeated herein.

In some embodiments, if the processor calculates the first mixed ignition temperature coefficient to be 1.22, the second mixed ignition temperature coefficient to be 1.12, the first mixed burnout temperature coefficient to be 1.22, the second mixed burnout temperature coefficient to be 1, the first mixed combustion characteristic coefficient to be 0.68, and the second mixed combustion characteristic coefficient to be 0.9 through the previously described manner, the equations for calculating the combustion characteristics of the mixture to be tested may be expressed as follows: for the mixture to be tested, the ignition temperature T=1.22×Tₘ×X+1.12×Tₙ×(1−X); the burnout temperature t=1.22×tₘ×X+1×tₙ×(1−X); and the composite combustion characteristic index B=0.68×Bₘ×X+0.9×Bₙ×(1−X).

In some embodiments of the present disclosure, the combustion characteristic of the coal to be tested and the combustion characteristic of the gasification slag to be tested are determined based on the first combustion coefficient, the second combustion coefficient, and the target basic data, and the combustion characteristic of the mixture to be tested is determined by combining the mixing ratio, which takes influence of different basic data on a combustion situation into account, thereby making the finally determined combustion characteristic of the mixture to be tested more accurate.

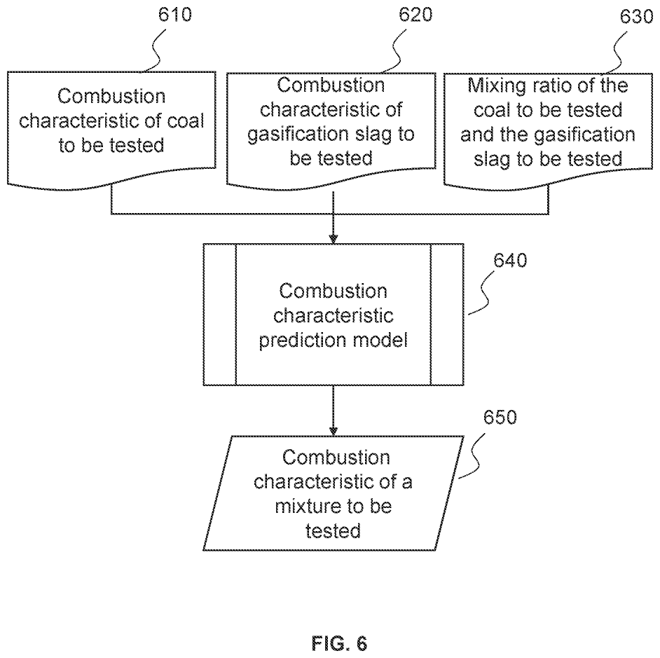

In some embodiments, the processor may determine the combustion characteristic of the mixture to be tested by processing the combustion characteristic of the coal to be tested, the combustion characteristic of the gasification slag to be tested, and the mixing ratio through a combustion characteristic prediction model. More descriptions on the embodiment may be found in FIG. 6 and the relevant descriptions thereof.

In some embodiments of the present disclosure, the target basic data is determined based on the first basic data and the second basic data, and the combustion characteristic of the mixture to be tested is determined by combining the second basic data and the mixing ratio, which uses the first basic data stored in a storage unit, which helps to reduce computational complexity.

FIG. 3 is a flowchart illustrating an exemplary process for determining a correction coefficient according to some embodiments of the present disclosure. In some embodiments, the process 300 may be executed by a processor. As shown in FIG. 3, the process 300 may include the operations 310, 320, 330, and 340.

In 310, determining an importance of each piece of basic data of target basic data.

More descriptions regarding the target basic data may be found in FIG. 2 and the related descriptions thereof.

The importance refers to a parameter that reflects an impact of each piece of basic data on the correction coefficient.

In some embodiments, the processor may determine the importance of each piece of basic data based on a type of the basic data through a preset data comparison table. The preset data comparison table records importances corresponding to different types of basic data.

In some embodiments, the processor may determine a basic importance of the each piece of basic data, determine an importance adjustment amount of the each piece of basic data based on a data distribution of the each piece of basic data of a basic database, and determine the importance of the each piece of basic data based on the basic importance of the each piece of basic data and the importance adjustment amount.

More descriptions about the basic database may be found in FIG. 1 and its related description.

The basic importance refers to a preset initial importance.

In some embodiments, the basic importance may be determined based on an impact of each type of basic data on a combustion characteristic. For example, if contents of carbon C, hydrogen H, and oxygen O have a great impact on the combustion characteristic, a great basic importance may be set to the carbon C, hydrogen H, and oxygen O. As another example, if contents of nitrogen N and sulfur S have a small impact on the combustion characteristic, a small basic importance may be set to the nitrogen N and sulfur S.

The data distribution refers to a parameter that reflects a distribution of the basic data.

In some embodiments, based on each same type of basic data of the basic database, the processor may calculate a variance of the same type of basic data and determine the variance as the data distribution of the basic data. For example, for the carbon, the processor may calculate a variance of carbon of all sample coal of the basic database and determine the variance as the data distribution corresponding to the carbon.

The importance adjustment amount refers to a parameter for adjusting the importance of the basic data.

In some embodiments, a storage device may pre-store corresponding relationships between the data distributions and the importance adjustment amounts of different types of basic data. The processor may access the storage device based on the type of and the data distribution of the determined basic data to determine the importance adjustment amount of the basic data through the corresponding relationships.

In some embodiments, the processor may sum the basic importance and the importance adjustment amount corresponding to the basic data and determine the sum as the importance of the basic data.

In some embodiments of the present disclosure, the processor determines the importance of the basic data based on the basic importance and the importance adjustment amount, which not only takes influence of different types of basic data on the combustion characteristic, but also takes the data distributions of different types of basic data of the basic database into account, thereby making a finally determined importance of the basic data more reasonable and accurate.

In 320, determining a verification interval size and a verification step size of the each piece of basic data based on the importance of the each piece of basic data.

The verification interval size refers to an amount of data contained in a single verification interval.

The verification step size refers to an amount of data between two adjacent verification intervals.

In some embodiments, the processor may determine the verification interval size and the verification step size of each piece of basic data based on the importance of each piece of basic data according to a preset rule. For example, the larger the importance of the basic data, the larger the verification interval size, and the smaller the verification step size.

In 330, constructing an orthogonal table by a preset algorithm based on the verification interval size and the verification step size of the each piece of basic data and controlling one or more testing units to conduct an orthogonal experiment based on the orthogonal table.

In some embodiments, the processor may construct the orthogonal table through an orthogonal table construction software or other suitable means based on the verification interval size and the verification step size of each piece of basic data. In some embodiments, the preset algorithm may be stored in one or more storage units.

In 340, determining the correction coefficient corresponding to the each piece of basic data based on an experimental result of the orthogonal experiment.

More descriptions about the correction coefficient may be found in FIG. 2 and the related descriptions thereof.

In some embodiments, the processor may calculate an average of all correction coefficients in the experimental result, and determine a group of correction coefficients that are closest to the average as the correction coefficients corresponding to the basic data.

In some embodiments of the present disclosure, the processor determines the verification interval size and the verification step size of each piece of basic data based on the importance of the each piece of basic data, conducts the orthogonal experiment, and determines the correction coefficient corresponding to the each piece of basic data, which not only reduces the computational complexity but also improves the accuracy of a finally determined correction coefficient.

It should be noted that the descriptions of the process 100 and the process 300 are provided merely for illustration and example and does not limit the scope of the present disclosure. Those skilled in the art may make various modifications and changes to the process 100 and the process 300 under the guidance of the present disclosure. However, such modifications and changes are still within the scope of the present disclosure.

FIG. 4 is a schematic diagram illustrating an exemplary first coefficient determination model according to some embodiments of the present disclosure.

The first coefficient determination model 420 may be a machine learning model for determining a first combustion coefficient, for example, a deep neural network model (DNN), etc. In some embodiments, the first coefficient determination model may be stored in one or more storage units.

In some embodiments, an input of the first coefficient determination model may include a first characteristic distance distribution 410 of coal to be tested, and an output of the first coefficient determination model may include a first combustion coefficient 430. More descriptions about the first combustion coefficient may be found in FIG. 2 and the related descriptions thereof.

The first characteristic distance distribution refers to a distribution of differences between basic data of the coal to be tested m and basic data of the target coal α. In some embodiments, the first characteristic distance distribution may be represented in a form of a vector. More descriptions about the target coal may be found in FIG. 2 and the related descriptions thereof.

In some embodiments, the processor may calculate a difference between a same type of basic data corresponding to the coal to be tested m and the target coal α and determine a vector representing differences of all basic data as the first characteristic distance distribution. In some embodiments, the processor may calculate the difference between the same type of basic data corresponding to the coal to be tested m and the target coal a and input the differences of all basic data into an embedding layer for processing to determine the first characteristic distance distribution.

In some embodiments, the first coefficient determination model may be obtained by training through a first loss function based on a first training set.

In some embodiments, the first loss function is constructed based on the output of the first coefficient determination model and a first label of a first training sample in the first training set.

In some embodiments, the first training set includes a plurality of first training samples and the first labels corresponding to the plurality of first training samples. The first training sample may include a sample first characteristic distance distribution, and the first label may include a sample first combustion coefficient.

In some embodiments, the first training set may be constructed based on data of a basic database and stored in the one or more storage units.

In some embodiments, the processor may randomly select two varieties of sample coal from the basic database and determine one of the two varieties of sample coal as a first sample coal and the other as a second sample coal. The processor calculates the first characteristic distance distribution of the two varieties of sample coal and determine the first characteristic distance distribution of the two varieties of sample coal as the sample first characteristic distance distribution.

In some embodiments, the processor may determine a combustion characteristic of the first sample coal as the combustion characteristic of the coal to be tested and a combustion characteristic of the second sample coal as the combustion characteristic of the target coal. The processor brings the combustion characteristic of the coal to be tested and the combustion characteristic of the target coal into equations for calculating the combustion characteristic to calculate the first combustion coefficient and determines the calculated first combustion coefficient as the sample first combustion coefficient. More descriptions about the combustion characteristic may be found in FIG. 2 and the related descriptions thereof.

In some embodiments of the present disclosure, the first training set may be constructed based on the data of the basic database, which is conducive to solving the problem that it is difficult to obtain the first training sample and the first label and enriches an amount of data of the first training set.

In some embodiments of the present disclosure, the first combustion coefficient may be determined by processing the first characteristic distance distribution of the coal to be tested through the first coefficient determination model, which takes influence of a plurality of factors into account at the same time, makes the determination of the first combustion coefficient more efficient and accurate, and avoids errors caused by manual determination.

FIG. 5 is a schematic diagram illustrating an exemplary second coefficient determination model according to some embodiments of the present disclosure.

The second coefficient determination model 520 may be a machine learning model for determining a second combustion coefficient, for example, a deep neural network (DNN) model, etc. In some embodiments, the second coefficient determination model is stored in one or more storage units.

In some embodiments, an input of the second coefficient determination model may include a second characteristic distance distribution 510 of gasification slag to be tested, and an output of the second coefficient determination model may include a second combustion coefficient 530. More descriptions about the second combustion coefficient may be found in FIG. 2 and the related descriptions thereof.

The second characteristic distance distribution refers to a distribution of differences between basic data of the gasification slag to be tested n and the target gasification slag β. In some embodiments, the second characteristic distance distribution may be represented in a form of a vector. More descriptions about the target gasification slag may be found in FIG. 2 and the related descriptions thereof. The determination manner of the second characteristic distance distribution is similar to the determination manner of the first characteristic distance distribution. The determination and more descriptions of the second characteristic distance distribution, may be found in the relevant descriptions of the first characteristic distance distribution in FIG. 4.

In some embodiments, the second coefficient determination model may be obtained by training through a second loss function based on a second training set.

In some embodiments, the second loss function is constructed based on the output of the second coefficient determination model and a second label of a second training sample in the second training set.

In some embodiments, the second training set includes a plurality of second training samples and the second labels corresponding the plurality of second training samples. The second training sample may include a sample second characteristic distance distribution, and the second label may include a sample second combustion coefficient.

In some embodiments, the second training set may be constructed based on data from a basic database and stored in the one or more storage units. The construction manner of the second training set is similar to the construction manner of the first training set. More descriptions about the construction of the second training set may be found in the relevant descriptions of the first training set in FIG. 4.

In some embodiments of the present disclosure, the second training set may be constructed based on the data from the basic database, which is conducive to solving the problem that it is difficult to obtain the second training samples and the second label and which enriches an amount of data of the second training set.

In some embodiments of the present disclosure, the second combustion coefficient may be determined by processing the second characteristic distance distribution of the gasification slag to be tested through the second coefficient determination model, which takes influence of a plurality of factors into account at the same time, makes the determination of the second combustion coefficient more efficient and accurate, and avoids errors caused by manual determination.

FIG. 6 is a schematic diagram illustrating an exemplary combustion characteristic prediction model according to some embodiments of the present disclosure.

The combustion characteristic prediction model 640 may be a machine learning model for determining a combustion characteristic of a mixture to be tested, for example, a deep neural network (DNN) model, etc. In some embodiments, the combustion characteristic prediction model is stored in one or more storage units.

In some embodiments, an input of the combustion characteristic prediction model may include a combustion characteristic 610 of coal to be tested, a combustion characteristic 620 of gasification slag to be tested, and a mixing ratio 630 of the coal to be tested and the gasification slag to be tested, and an output of the combustion characteristic prediction model may include a combustion characteristic 650 of the mixture to be tested. More descriptions about the mixing ratio and the combustion characteristic may be found in FIG. 1 and the related descriptions.

In some embodiments, the combustion characteristic prediction model may be obtained by training through a third loss function based on a third training set.

In some embodiments, the third loss function is constructed based on the output of the combustion characteristic prediction model and a third label of a third training sample in the third training set. The third training set is stored in the one or more storage units.

In some embodiments of the present disclosure, the combustion characteristic prediction model may be trained by constructing the third training set and, and a training process may be constrained through the third loss function, so that the combustion characteristic prediction model becomes more accurate, and a predicted combustion characteristic outputted from the combustion characteristic prediction model is more reliable.

In some embodiments, the third training sample may include a combustion characteristic of sample coal, a combustion characteristic of sample gasification slag, and a sample mixing ratio. The third label may include a combustion characteristic of a sample mixture.

In some embodiments, the third training set may include a real sample and a theoretical sample.

The real sample refers to data measured through an experiment.

In some embodiments, the real sample is constructed based on actual measured data of a mixture obtained by mixing sample coal and sample gasification slag in a preset mixing ratio. The sample coal and sample gasification slag is randomly selected from a basic database. For example, the processor may control one or more testing units to conduct a combustion characteristic test on the mixture to determine the real sample. The mixture is obtained by mixing the randomly selected sample coal and sample gasification slag.

The random selecting may include Gaussian random, Bernoulli random, etc. The theoretical sample refers to a sample generated based on a theoretical calculation equation for determining the combustion characteristic of the mixture to be tested. For example, the theoretical sample may include data calculated based on the basic data of the sample coal and the sample gasification slag through the calculation equations provided in the FIGS. 1-3. In some embodiments, coefficients of the theoretical calculation equation may be obtained by fitting based on the real sample.

In some embodiments, the training of the combustion characteristic prediction model by the processor further includes determining a learning rate of a current round of iteration based on a type (e.g., the real sample or the theoretical sample) of the third training sample at the time of each round of iteration.

The learning rate is configured to reflect a magnitude of update of a current round of iteration to a network model parameter. The larger the learning rate, the greater the magnitude of update of the current round of iteration to the network model parameter.

In some embodiments, the processor may determine the learning rate of the current round of iteration based on the type of the third training sample through a second preset rule. For example, a larger learning rate may be set when the third training sample is the real sample, and a smaller learning rate may be set when the third training sample is the theoretical sample. In some embodiments, the second preset rule may include the learning rate when the network model parameter is updated based on the real sample being greater than the learning rate when the network model parameter is updated based on the theoretical sample.

In some embodiments of the present disclosure, the real sample and the theoretical sample may be constructed and learning rates of different magnitudes may be set, which not only helps to enrich data of the third training set but also takes into account an error that exists in the theoretical sample, so that the output of an ultimately trained combustion characteristic prediction model can be more accurate.

In some embodiments of the present disclosure, the combustion characteristic of the mixture may be predicted by processing the combustion characteristic of the coal to be tested, the combustion characteristic of the gasification slag to be tested, and the mixing ratio of the coal to be tested and gasification slag to be tested in a comprehensive manner through the combustion characteristic prediction model, which takes influence of a plurality of factors into account at the same time, makes a prediction result of the combustion characteristic of the mixture more accurate and reliable, and avoids errors caused by manual determination.

Merely by way of example, in some embodiments, for a mixture to be tested obtained by mixing certain gasification slag to be tested and certain coal to be tested, the processor may calculate and determine the combustion characteristic of the mixture to be tested by implementing the following operations.

In S1, 10 varieties of representative gasification slag and 5 varieties of representative coal as experimental samples are selected based on factors such as a moisture content M, a volatile matter V, and a fixed carbon FC. Different first basic data of each variety of sample gasification slag and each variety of sample coal sample are obtained by controlling one or more testing units to perform an industrial analysis, an elemental analysis, a calorific value Q and combustion characteristic test on each sample, respectively. A basic database of the 10 varieties of gasification slag and 5 varieties of coal is established, and the basic database is stored in one or more storage units.

In S2, second basic data of the coal to be tested m and the gasification slag to be tested n are obtained by controlling one or more testing units to perform the test on mixtures to be tested obtained by mixing an unknown variety of gasification slag to be tested (denoted as n) and an unknown variety of coal to be tested (denoted as m) with different ratios according to the testing manner in S1.

A sum of squares of differences between the first basic data corresponding to the coal to be tested m and the first basic data corresponding to any variety of sample coal in the basic database is calculated, and first basic data with a smallest sum of squares is selected as target basic data of the coal to be tested m. Similarly, a sum of squares of differences between the first basic data corresponding to the gasification slag to be tested n and the first basic data corresponding to any variety of sample gasification slag in the basic database is calculated, and first basic data with a smallest sum of squares is selected as target basic data of the gasification slag to be tested n. Experimental samples providing the two sets of target basic data are referred to as target basic samples. The target basic sample corresponding to the coal to be tested m is denoted as the target coal α and the target basic sample corresponding to the gasification slag to be tested n is denoted as the target gasification slag β.

In S3, the first combustion coefficient Kₘ of the coal to be tested m based on the target basic data of the target coal α and the second basic data of the coal to be tested m is calculated. The first combustion coefficient Kₘ is determined by calculating a sum of squares of differences between basic data values of the target coal α and basic data values of the coal to be tested m and multiplying the squares by correction coefficients corresponding to various pieces of basic data, respectively. The second combustion coefficient Kₙ of the gasification slag to be tested n based on the target basic data of the target gasification slag β and the second basic data of the gasification slag to be tested n is calculated. The second combustion coefficient Kₙ is determined by calculating a sum of squares of differences between basic data values of the target gasification slag β and basic data values of the gasification slag to be tested n and multiplying the squares by a correction coefficients corresponding to various pieces of basic data, respectively.

In S4, the combustion characteristic of the coal to be tested m and the combustion characteristic of the gasification slag to be tested n are calculated through the following equations. The combustion characteristic includes the ignition temperature T, the burnout temperature t, and the composite combustion characteristic index B.

The ignition temperature Tₘ of the coal to be tested m:

$${T}_{m} = {T}_{α} \times \frac{{K}_{m}}{10} ,$$

where T_{α} denotes the ignition temperature of the target coal α.

The burnout temperature tₘ of the coal to be tested m:

$${t}_{m} = {t}_{α} \times \frac{{K}_{m}}{15} ,$$

where t_{α} denotes the burnout temperature of the target coal α.

The composite combustion characteristic index Bₘ of the coal to be tested m:

$${B}_{m} = {B}_{α} \times \frac{{K}_{m}}{110} ,$$

where B_{α} denotes the composite combustion characteristic index of the target coal α.

The ignition temperature Tₙ of the gasification slag to be tested n:

$${T}_{n} = {T}_{β} \times \frac{{K}_{n}}{22} ,$$

where T_{β} denotes the ignition temperature of the target gasification slag β.

The Burnout temperature tₙ of the gasification slag to be tested n:

$${t}_{n} = {t}_{β} \times \frac{{K}_{n}}{24} ,$$

where t_{β} denotes the burnout temperature of the target gasification slag β.

The composite combustion characteristic index Bₙ of the gasification slag to be tested n:

$${B}_{n} = {B}_{β} \times \frac{{K}_{n}}{130} ,$$

where B_{β} denotes the composite combustion characteristic index of the target gasification slag β.

In S5, assuming a mass fraction of the coal to be tested m in the mixture to be tested is X, for the mixture to be tested,
the ignition temperature T=1.22×Tₘ×X+1.12×Tₙ×(1−X);
the burnout temperature t=1.22×tₘ×X+tₙ×(1−X); and
the composite combustion characteristic index B=0.68×Bₘ×X+0.9×Bₙ×(1−X).

Merely by way of example, using the method for determining the combustion characteristic of the mixture of gasification slag and coal provided in some embodiments of the present disclosure, for a certain mixture to be tested obtained by mixing the certain gasification slag to be tested and the certain coal to be tested, properties of the coal to be tested m and gasification slag to be tested n are measured by the processor as follows (all values are based on air-dried basis):

| | | | | | | | | | | Q |
|---|---|---|---|---|---|---|---|---|---|---|
| | M | V | A | FC | C | H | O | N | S | (MJ/Kg) |
| Coal to be | 0.13% | 34.20% | 6.27% | 59.40% | 76.80% | 5.00% | 5.24% | 1.10% | 1.80% | 30.30 |
| tested m | | | | | | | | | | |
| Gasification | 0.20% | 2.10% | 54.30% | 43.40% | 47.80% | 0.50% | 4.89% | 0.30% | 1.90% | 16.14 |
| slag to be | | | | | | | | | | |
| tested n | | | | | | | | | | |

Following the operations in FIG. 2, the processor calculates fₘᵢₙ=0.25 and hₘᵢₙ=0.24. Correspondingly, properties of the target coal a and target gasification slag β are as follows (all values are based on air-dried basis):

| | M | V | A | FC | C | H | O | N | S | Q |
|---|---|---|---|---|---|---|---|---|---|---|
| Target coal α | 3.36% | 24.29% | 35.66% | 36.69% | 45.22% | 2.80% | 11.20% | 0.46% | 1.30% | 21.01 MJ/Kg |
| Target | 0.54% | 2.64% | 85.48% | 11.34% | 40.34% | 2.42% | 5.62% | 0.25% | 1.60% | 3.88 MJ/Kg |
| gasification | | | | | | | | | | |
| slag β | | | | | | | | | | |

The ignition temperature T, the burnout temperature t, and the composite combustion characteristic index B of the target coal α and target gasification slag β are as follows:

| | Ignition | Burnout | Composite combustion |
|---|---|---|---|
| | temperature | temperature | characteristic index B × 10⁻⁷ |
| | T (° C.) | t (° C.) | %² (min² · ° C.³)⁻¹ |
| Target Coal | 300.33 | 565.71 | 27.0 |
| α Target | | | |
| Gasification | 529.59 | 640.74 | 3.5 |
| slag β | | | |

The processor continues the calculation according to the data and the equations for calculating the combustion coefficients as provided in FIG. 2 and obtains the following results: for the coal to be tested m, the first combustion coefficient Kₘ=15.08447, the ignition temperature Tₘ=453.0317, the burnout temperature tₘ=568.8955, and the composite combustion characteristic index Bₘ=3.7026; for the gasification slag to be tested n, the second combustion coefficient Kₙ=25.97, the ignition temperature Tₙ=625.0897, the burnout temperature tₙ=693.2595, and the composite combustion characteristic index Bₙ=0.6991.

For mixtures to be tested obtained by mixing the gasification slag to be tested and the coal to be tested with different ratios, the combustion characteristic data is calculated using the manners of the embodiments of the present disclosure (see FIGS. 1-6) and shown in Table a as follows:

TABLE a
Combustion characteristic data calculated using the
manners of the embodiments of the present disclosure

| | | | | | | Single |
|---|---|---|---|---|---|---|
| | Single | | | | | gasification |
| | coal | Ratio 1 | Ratio 2 | Ratio 3 | Ratio 4 | slag |
| Coal to be | 1.00 | 0.75 | 0.50 | 0.25 | 0.20 | — |
| tested m | | | | | | |
| Gasification slag | — | 0.25 | 0.50 | 0.75 | 0.80 | 1.00 |
| to be tested n | | | | | | |
| Ignition | 453.0317 | 477.19 | 461.71 | 446.23 | 443.13 | 625.0897 |
| temperature T | | | | | | |
| Burnout | 568.8955 | 698.98 | 692.92 | 686.86 | 685.65 | 693.2595 |
| temperature t | | | | | | |
| Composite | 3.7026 | 2.02 | 1.55 | 1.08 | 0.99 | 0.9661 |
| combustion | | | | | | |
| characteristic | | | | | | |
| index B × 10⁻⁷ | | | | | | |

The mixture to be tested obtained by mixing the gasification slag to be tested and the coal to be tested with the ratios are subjected to experimental measurements, and actual combustion characteristic data is obtained and shown in Table b as follows:

TABLE b
Actual combustion characteristic data obtained by experimental measurements

| | | | | | | Single |
|---|---|---|---|---|---|---|
| | Single | | | | | gasification |
| | coal | Ratio 1 | Ratio 2 | Ratio 3 | Ratio 4 | slag |
| Coal to be | 1.00 | 0.75 | 0.50 | 0.25 | 0.20 | — |
| tested m | | | | | | |
| Gasification slag | — | 0.25 | 0.50 | 0.75 | 0.80 | 1.00 |
| to be tested n | | | | | | |
| Ignition | 439.10 | 447.50 | 456.60 | 464.50 | 465.70 | 601.60 |
| temperature T | | | | | | |
| Burnout | 577.90 | 703.20 | 694.70 | 684.00 | 684.50 | 680.80 |
| temperature t | | | | | | |
| Composite | 3.66 | 1.98 | 1.45 | 1.05 | 1.04 | 0.68 |
| combustion | | | | | | |
| characteristic | | | | | | |
| index B × 10⁻⁷ | | | | | | |

By comparing the data in table a and table b, it may be observed that when the combustion characteristics of mixtures with different ratios are calculated through the method for determining the combustion characteristic of the mixture of gasification slag and coal provided by some embodiments of the present disclosure, the majority of calculated errors are within 5%, and few data may have errors exceeding 5% but not exceeding 10%, which indicates that the method for determining the combustion characteristic of the mixture of gasification slag and coal provided by some embodiments of the present disclosure is an accurate and efficient method for calculating the combustion characteristic of the mixture of gasification slag and coal.

One or more embodiments of the present disclosure provide a system for determining a combustion characteristic of a mixture of gasification slag and coal. The combustion characteristic includes at least one of an ignition temperature, a burnout temperature, or a composite combustion characteristic index. The system includes: a first obtaining module configured to obtain first basic data of sample coal and sample gasification slag, the first basic data including at least one of a moisture content, a volatile matter, or a fixed carbon; a second obtaining module configured to obtain second basic data of coal to be tested and gasification slag to be tested in a mixture to be tested and a mixing ratio of the mixture to be tested; and a determination module configured to determine a combustion characteristic of the mixture to be tested based on the first basic data, the second basic data, and the mixing ratio.

One or more embodiments of the present disclosure provide a non-transitory computer-readable storage medium that stores computer instructions. When reading the computer instructions in the storage medium, a computer implements the method for determining a combustion characteristic of a mixture of gasification slag and coal according to any of the embodiments.

The basic concepts have been described above, and it is clear that the above detailed disclosure is intended as an example only for those skilled in the art and does not constitute a limitation of the present disclosure. Although not explicitly stated herein, there are various modifications, improvements, and amendments that may be made to the present disclosure by those skilled in the art. Such modifications, improvements, and amendments are suggested in the present disclosure, so such modifications, improvements, and amendments remain within the spirit and scope of the exemplary embodiments of the present disclosure.

Also, the present disclosure uses specific words to describe embodiments of the present disclosure. For example, “one embodiment,” “an embodiment,” and/or “some embodiments” mean that a certain feature, structure, or characteristic is connected with at least one embodiment of the present disclosure. Therefore, it should be emphasized and noted that “one embodiment” or “an embodiment” or “an alternative embodiment” mentioned twice or more in different places in the present disclosure does not necessarily refer to the same embodiment. In addition, certain features, structures, or characteristics of one or more embodiments of the present disclosure may be suitably combined.

Additionally, unless explicitly stated in the claims, the order of processing elements and sequences, the use of numerical or alphabetical values, or other nomenclature used in the present disclosure is not intended to limit the sequence of the processes and methods described herein. While various examples have been discussed in the foregoing disclosure to illustrate some presently considered useful embodiments of the present disclosure, it should be understood that such details are provided for illustrative purposes only. The appended claims are not limited to the disclosed embodiments but are intended to cover all modifications and equivalent combinations falling within the scope and spirit of the embodiments described in the present disclosure. For instance, although the system components described above may be implemented through hardware devices, they may also be realized solely through software solutions, such as installing the described system on existing servers or mobile devices.

Similarly, it should be noted that, for the sake of simplifying the presentation of the embodiments disclosed in the present disclosure and aiding in the understanding of one or more embodiments of the present disclosure, features from various embodiments, drawings, or descriptions may have been combined into a single embodiment. However, this way of disclosure does not imply that more features are required for the subject matter of the claims than are explicitly mentioned in the claims. In fact, the features of the embodiments are fewer than all the features disclosed in any single embodiment described above.

Some embodiments may use numbers to describe the quantity of components or attributes. It should be understood that such numbers used for the description of embodiments may be modified with qualifying terms such as “about,” “approximately,” or “substantially.” Unless otherwise stated, these qualifying terms indicate that the numbers allow for a variation of ±20%. Accordingly, in some embodiments, the numerical parameters used in the present disclosure and claims are approximate values, which may vary depending on specific embodiments' requirements. In some embodiments, numerical parameters should be construed to include the specified number of significant digits and the means of general rounding off. Although the numerical ranges and parameters provided in some embodiments of the present disclosure are approximate values, the setting of such numerical values in specific embodiments is as accurate as possible within the feasible range.

With respect to each patent, patent application, published patent application, and other materials referenced in the present disclosure, such as articles, books, manuals, publications, documents, etc., the entirety of each is hereby incorporated by reference into the present disclosure. However, except for application history documents that are inconsistent with or conflict with the content of the present disclosure, any documents that limit the broadest scope of the claims of the present disclosure (currently or subsequently added) are excluded. It should be noted that if there are any inconsistencies or conflicts between the descriptions, definitions, and/or terms used in the annexed materials and the content of the present disclosure, the descriptions, definitions, and/or terms used in the present disclosure shall prevail.

In closing, it should be understood that the embodiments described in the present disclosure are intended to illustrate the principles of the embodiments disclosed herein. Other variations may also fall within the scope of the present disclosure. Therefore, by way of example and not limitation, alternative configurations of the embodiments disclosed in the present disclosure may be considered consistent with the teachings of the present disclosure. Accordingly, the embodiments described in the present disclosure are not limited to the explicitly introduced and described embodiments in the present disclosure.

What is claimed is:

1. A method for determining a combustion characteristic of a mixture of gasification slag and coal, implemented by a processor, wherein the combustion characteristic includes at least one of an ignition temperature, a burnout temperature, or a composite combustion characteristic index, comprising:
obtaining first basic data of sample coal and sample gasification slag, the first basic data including at least one of a moisture content, a volatile matter, or a fixed carbon;
obtaining second basic data of coal to be tested and gasification slag to be tested in a mixture to be tested and a mixing ratio of the mixture to be tested; and
determining a combustion characteristic of the mixture to be tested based on the first basic data, the second basic data, and the mixing ratio.

2. The method of claim 1, wherein the obtaining first basic data of sample coal and sample gasification slag includes:
selecting a first quantity and variety of coal as the sample coal and a second quantity and variety of gasification slag as the sample gasification slag, and determining the first basic data of the sample coal and the sample gasification slag by controlling one or more testing units to perform at least one of an industrial analysis, an elemental analysis, a calorific value, and combustion characteristic test on the sample coal and the sample gasification slag; and
establishing a basic database of the sample coal and the sample gasification slag based on the first basic data, and storing the basic database in one or more storage units.

3. The method of claim 2, wherein the industrial analysis includes determining the moisture content, an ash content, the volatile matter, and the fixed carbon, and the elemental analysis includes determining carbon, hydrogen, oxygen, nitrogen, and sulfur.

4. The method according to claim 1, wherein the obtaining second basic data of coal to be tested and gasification slag to be tested in a mixture to be tested includes:
determining the second basic data of the coal to be tested and the gasification slag to be tested by controlling one or more testing units to perform at least one of an industrial analysis, an elemental analysis, a calorific value, and combustion characteristic test on the coal to be tested and the gasification slag to be tested.

5. The method of claim 1, wherein the determining a combustion characteristic of the mixture to be tested based on the first basic data, the second basic data, and the mixing ratio includes:
determining target basic data of the coal to be tested and the gasification slag to be tested based on the first basic data and the second basic data; and
determining the combustion characteristic of the mixture to be tested based on the target basic data, the second basic data, and the mixing ratio.

6. The method of claim 5, wherein the determining the combustion characteristic of the mixture to be tested based on the target basic data, the second basic data, and the mixing ratio includes:
determining a first combustion coefficient of the coal to be tested and a second combustion coefficient of the gasification slag to be tested based on the target basic data and the second basic data;
determining a combustion characteristic of the coal to be tested and a combustion characteristic of the gasification slag to be tested based on the first combustion coefficient, the second combustion coefficient, and the target basic data; and
determining the combustion characteristic of the mixture to be tested based on the combustion characteristic of the coal to be tested, the combustion characteristic of the gasification slag to be tested, and the mixing ratio.

7. The method of claim 6, wherein the first combustion coefficient and the second combustion coefficient are related to a correction coefficient.

8. The method of claim 7, wherein correction coefficients corresponding to a same type of basic data of the coal to be tested and the gasification slag to be tested are the same.

9. The method of claim 7, wherein determining the correction coefficient includes:
determining an importance of each piece of basic data of the target basic data;
determining a verification interval size and a verification step size of the each piece of basic data based on the importance of the each piece of basic data;
constructing an orthogonal table by a preset algorithm based on the verification interval size and the verification step size of the each piece of basic data, and controlling one or more testing units to conduct an orthogonal experiment based on the orthogonal table, the preset algorithm being stored in one or more storage units; and
determining the correction coefficient corresponding to the each piece of basic data based on an experimental result of the orthogonal experiment.

10. The method of claim 9, wherein the determining an importance of each piece of basic data of the target basic data includes:
determining a basic importance of the each piece of basic data;
determining an importance adjustment amount of the each piece of basic data based on a data distribution of the each piece of basic data of a basic database; and
determining the importance of the each piece of basic data based on the basic importance of the each piece of basic data and the importance adjustment amount.

11. The method of claim 6, wherein determining the first combustion coefficient includes:
determining the first combustion coefficient by processing a first characteristic distance distribution of the coal to be tested through a first coefficient determination model, the first coefficient determination model being a machine learning model, and the first coefficient determination model being stored in one or more storage units.

12. The method of claim 11, wherein the first coefficient determination model is obtained by training through a first loss function based on a first training set, the first loss function is constructed based on an output of the first coefficient determination model and a first label of a first training sample of the first training set, the first training set is constructed based on data of the basic database, and the first training set is stored in the one or more storage units.

13. The method of claim 6, wherein determining the second combustion coefficient includes:
determining the second combustion coefficient by processing a second characteristic distance distribution of the gasification slag to be tested through a second coefficient determination model, the second coefficient determination model being a machine learning model, and the second coefficient determination model being stored in one or more storage units.

14. The method of claim 13, wherein the second coefficient determination model is obtained by training through a second loss function based on a second training set, the second loss function is constructed based on an output of the second coefficient determination model and a second label of a second training sample of the second training set, the second training set is constructed based on data of the basic database, and the second training set is stored in the one or more storage units.

15. The method of claim 6, wherein the determining the combustion characteristic of the mixture to be tested based on the combustion characteristic of the coal to be tested, the combustion characteristic of the gasification slag to be tested, and the mixing ratio includes:
determining the combustion characteristic of the mixture to be tested by processing the combustion characteristic of the coal to be tested, the combustion characteristic of the gasification slag to be tested, and the mixing ratio through a combustion characteristic prediction model, the combustion characteristic prediction model being a machine learning model, and the combustion characteristic prediction model being stored in one or more storage units.

16. The method of claim 15, wherein the combustion characteristic prediction model is obtained by training through a third loss function based on a third training set, the third loss function being constructed based on an output of the combustion characteristic prediction model and a third label of a third training sample of the third training set, and the third training set being stored in the one or more storage units.

17. The method of claim 16, wherein the third training set includes a real sample and a theoretical sample, the real sample is constructed based on actual measurement data of a mixture obtained by mixing the sample coal and the sample gasification slag randomly selected from the basic database in a preset ratio, the theoretical sample is generated by a theoretical calculation equation, and a coefficient of the theoretical calculation equation is obtained by fitting based on the real sample; and
training the combustion characteristic prediction model further includes: determining a learning rate of a current round of iteration according to a type of the third training sample at the time of each round of iteration.

18. A system for determining a combustion characteristic of a mixture of gasification slag and coal, the combustion characteristic including at least one of an ignition temperature, a burnout temperature, or a composite combustion characteristic index, comprising:
a first obtaining module configured to obtain first basic data of sample coal and sample gasification slag, the first basic data including at least one of a moisture content, a volatile matter, or a fixed carbon;
a second obtaining module configured to obtain second basic data of coal to be tested and gasification slag to be tested in a mixture to be tested and a mixing ratio of the mixture to be tested; and
a determination module configured to determine a combustion characteristic of the mixture to be tested based on the first basic data, the second basic data, and the mixing ratio.

19. The system of claim 18, wherein the determination module is further configured to:
determine target basic data of the coal to be tested and the gasification slag to be tested based on the first basic data and the second basic data; and
determine the combustion characteristic of the mixture to be tested based on the target basic data, the second basic data, and the mixing ratio.

20. A non-transitory computer-readable storage medium storing computer instructions, wherein when reading the computer instructions in the storage medium, a computer executes the method for determining a combustion characteristic of a mixture of gasification slag and coal of claim 1.
